(19) United States
(12) Patent Application Publication
(10) Pub. No.: US 2019/0387983 A1
(43) Pub. Date: Dec. 26, 2019

(54) PORTABLE, PEDIATRIC MEDICAL DIAGNOSTIC DEVICE

(71) Applicant: Michael SCRIPT, Fairfax Station, VA (US)

(72) Inventor: Michael SCRIPT, Fairfax Station, VA (US)

(21) Appl. No.: 16/530,825

(22) Filed: Aug. 2, 2019

Related U.S. Application Data
(63) Continuation-in-part of application No. 15/720,990, filed on Sep. 29, 2017.
Application No. 15/720,990 is a continuation-in-part of application No. 13/800,389, filed on Mar. 13, 2013, now abandoned.
(60) Provisional application No. 62/402,558, filed on Sep. 30, 2016.

(51) Int. Cl. A61B 5/0205; A61B 5/01; A61B 5/053; A61B 5/1455; A61B 5/00; A61B 5/0402; A61B 1/227

(52) U.S. Cl. CPC A61B 5/02055; A61B 5/01; A61B 5/0533; A61B 5/14551; A61B 5/024; A61B 5/0002; A61B 5/7475; A61B 5/0402; A61B 1/227; A61B 5/6831

(57) ABSTRACT

A hand-held pediatric medical diagnostic device includes a sensor module adapted to detect one or more corresponding conditions of a child. A gel material at least partially coats a contact surface on the diagnostic device. The area of the contact surface and the weight associated therewith are selected to substantially stabilize the sensor module during use. In one version, the pediatric device comprises a pediatric respiratory rate sensing device in which an accelerometer is connected to a portable, rechargeable power source.

### CROSS-REFERENCE TO RELATED APPLICATIONS

This application is a Continuation-in-Part of U.S. Ser. No. 13/800,389 filed Mar. 13, 2013 entitled “Portable, Pediatric Medical Diagnostic Device”, and is also a Continuation-in-Part of U.S. Ser. No. 15/720,990 filed Sep. 29, 2017 entitled “Portable, Pediatric Medical Diagnostic Device” which claims priority under 35 U.S.C. § 119(e) to U.S. Provisional Patent Application No. 62/402,558 filed Sep. 30, 2016 entitled “Portable, Pediatric Medical Diagnostic Device.

### FIELD

This disclosure relates to pediatric medical diagnostic devices and, in particular, to portables devices suitable for field use.

### BACKGROUND

The diagnosis of pediatric medical conditions in remote areas, in developing countries, in war-torn areas, or in other such “field” locations may present challenges to medical practitioners, relief- or aid-workers, or similar personnel. For example, typical hospital supplies may be unavailable or in short supply. Environmental factors may render traditional hospital devices inaccurate or nonfunctional, and sanitary conditions may be compromised.

In developing countries outside of hospital settings, or in other field situations, medical or relief personnel are often required to resort to counting, watches, or other basic techniques to take pediatric respiratory rates or other vital signs. Such basic or manual counting or timing methods are often not accurate or are often unable to be recorded or otherwise subsequently analyzed. Disposable medical supplies, including those related to diagnostic devices, may likewise be unavailable in remote or field applications, or in developing countries.

The age of children associated with pediatric diagnoses may cause them to squirm, cry or otherwise cause further challenges to obtaining accurate or useful readings by basic or manual counting and timing methods. All of the foregoing hampers effective treatment of medical conditions suffered by children in developing countries or in other less-than-optimal environments.

It would be desirable to address the foregoing drawbacks and disadvantages.

### SUMMARY

According to one implementation, a hand-held pediatric medical diagnostic device makes use of a sensor module to detect one or more corresponding medical conditions of a wearer. The sensor module has a contact surface which can be placed in operative contact with the wearer. The device includes a user interface to select a diagnostic program associated with the sensor module. The user interface includes a readout screen corresponding to the diagnostic procedure being performed.

In another implementation, the device comprises a respiratory sensing device with an accelerometer adapted to detect respiration of a wearer. A contact surface is operatively connected to the accelerometer and adapted to be placed in contact with the wearer's chest. A gel material at least partially coats the contact surface. One suitable gel material is silicone, but other gel materials, such as polyvinyl chloride and latex, are likewise suitable. The characteristics of the gel material and the area and weight associated with the contact surface act to substantially stabilize the accelerometer during use so as to reduce spurious signals.

### BRIEF DESCRIPTION OF THE DRAWINGS

The disclosure will be explained in greater detail hereinafter on the basis of exemplary implementations, and with reference to the drawing, in which:

FIG. 1 shows a front elevational view of one implementation of the present disclosure;

FIG. 2 shows a side elevational view of the implementation of FIG. 1;

FIG. 3 shows a rear elevational view of the implementation of FIGS. 1 and 2;

FIG. 4 shows another implementation of the present disclosure in partially schematic form; and

FIG. 5 shows yet another implementation of the present disclosure.

FIG. 6 shows one implementation of the sensor module;

FIG. 7 shows one implementation of vital sign readouts displayed as a sensor array;

FIG. 8 shows other implementations of vital sign readouts display;

FIGS. 9A-9B show one implementation of the sensor module connected to the banding system;

FIGS. 10A-10B show steps for attaching the sensor module to the banding system;

FIG. 11 shows one implementation of separating the sensor module and the banding system through an extension cable;

FIGS. 12A-12B show two wearing modes of the device;

FIGS. 13A-13C show one implementation of charging the sensor module by a charging system;

FIG. 14 shows two examples of vibration devices;

FIG. 15 is a diagram of hardware that can be used to implement an embodiment of the invention; and

FIG. 16 is a diagram of a chip set that can be used to implement an embodiment of the invention.

### DETAILED DESCRIPTION

Referring to the drawings, a portable pediatric medical diagnostic device has been implemented as a pediatric respiratory rate sensing device 21. Sensing device 21 makes use of an accelerometer 23, such as that disclosed in U.S. Pat. No. 7,554,445, the teachings of which are incorporated herein. In general terms, device 21 is used to detect the respiration rate of a wearer by placing a contact surface 25 against the chest of a wearer, such as on the sternum. The contact surface 25 is operatively connected to accelerometer 23, meaning that accelerometer 23 detects the relative raising and lowering of contact surface 25 from inhalation and exhalation of the wearer's chest over a period of time, thereby determining the respiration rate.

A gel material 27, such as silicone in this implementation, is disposed at least partially covering contact surface 25. Gel material 27 assists in holding device 21 in a desired position on the wearer and may also reduce shocks, jolts, or other movement of device 21 and its associated accelerometer 23 from causes other than pediatric respiration or other characteristics to be measured. Gel material 27 may likewise consist of polyvinyl chloride or latex. The area and weight associated with contact surface 25, as well as the characteristics of gel material 27, are selected to substantially stabilize accelerometer 23 during use and thereby reduce spurious signals.

For example, suitable implementations may have contact surface 25 with an area ranging from about 4 square inches to about 14 square inches, and a weight of about 130 grams to about 200 grams. In the illustrated implementation, gel material 27 has an area substantially corresponding to contact surface 25, a thickness of about ⅛ inch to ¼ inch, and a tackiness sufficient, under standard atmospheric conditions, to both adhere to the skin of the child in a variety of positions and be removable therefrom without substantially harming the skin of the child.

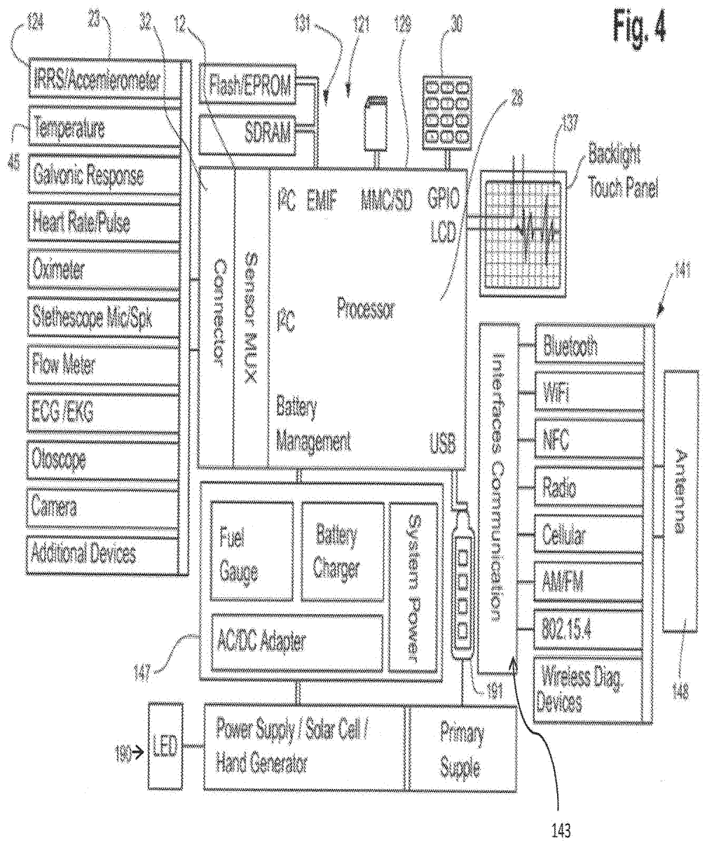

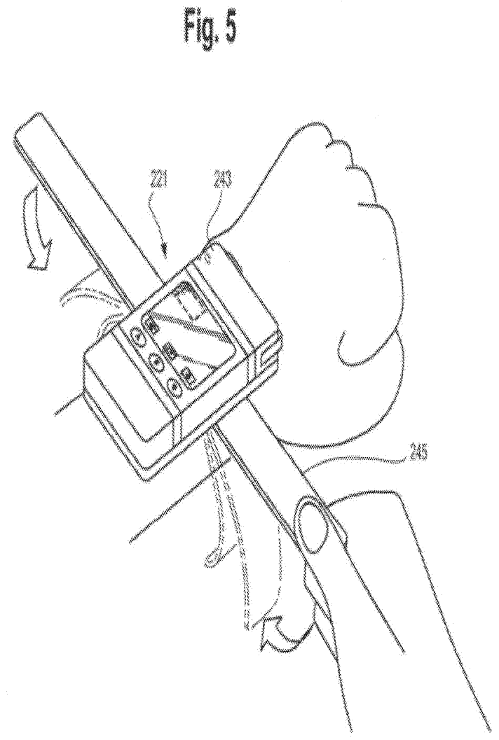

Operation of device 21 is accomplished through a suitable control system 29 housed in a user interface module 31. Control system 29 includes a suitable user interface and a processor 28 suitably programmed to process input from the user interface and from accelerometer 23. In this implementation, user interface module 31 and its associated control system 29 include a user interface having user input areas 33 in the form of user-activatable buttons 35 corresponding to respective modes of operation of device 21. User input areas 33 may also include a suitable keypad 30 (FIG. 4). In this implementation, control system 29 is configured to sense respiratory rates in children from newborn infants to age five, and buttons 35 allow operation of the device in the following three modes: infant, toddler, and child modes. It will be appreciated that other implementations of device 21 may be configured for other pediatric or child age ranges, including children age five and above. The provision of three, easily accessible buttons 35 corresponding to three respiratory programs, lends device 21 operational simplicity, which may be advantageous under adverse field conditions or environments. User interface module 31 further includes a screen 37 for displaying readouts associated with operation of device 21. Read-outs may assume any number of forms, such as numeric, graphical, color-coding, and audio or other visual indicators.

It would likewise be appreciated that interface module 31 may assume any number of alternative configurations, including having screen 37 comprise a touch screen with user-activatable areas, readouts, or any number of input and output functions, either in addition to or instead of depressible buttons 35.

Device 21 and its various components are powered by a portable, rechargeable power source 39 electrically connected to control system 29 and accelerometer 23. Power source 39 can be a replaceable battery, a rechargeable battery, a graphene capacitor, or a manually operable crank charger 41, as shown in this implementation.

Device 21 makes use of a suitable housing 43 inside of which one or more of the above-described components are carried or contained. In one implementation, contact surface 25 may comprise a lower surface of housing 43, and user interface module 31 may have its screen 37 viewable through an upper surface of housing 43. Power source 39 can be selectively connectable to housing 43, or removable therefrom, so that power source 39 can be replaced with an alternative power source, as needed. Physical attachment of power source 39 to housing 43 may also result in electrical connection of power source 39 to control system 29 and accelerometer 23 for suitable operation, such as through a suitable connector 32 (FIG. 4).

Accelerometer 23, in this implementation, is part of a sensor module 24 which optionally includes a temperature sensor 45. In order for sensor 45 to be operatively associated with the child, a suitable aperture 46 is formed in gel material 27, or other suitable conductive path may be provided. Sensor module 24 may be removably attached to user interface module 31. Power source 39, as discussed previously, may likewise assume the form of a user-swappable power module 47. In this implementation, then, sensor module 24, including accelerometer 23 and optional temperature sensor 45, and power module 47, comprising crank charger 41, are each user-detachable from module 31, to enable attachment of a second power module or a second sensor module, each potentially being different from the first such modules. As such, sensor module 24 with accelerometer 23 and option temperature sensor 45 can be substituted for another sensor module having one or more different diagnostic sensors, and, likewise, crank charger 41 can likewise be replaced with a battery, capacitor, or other suitable power source.

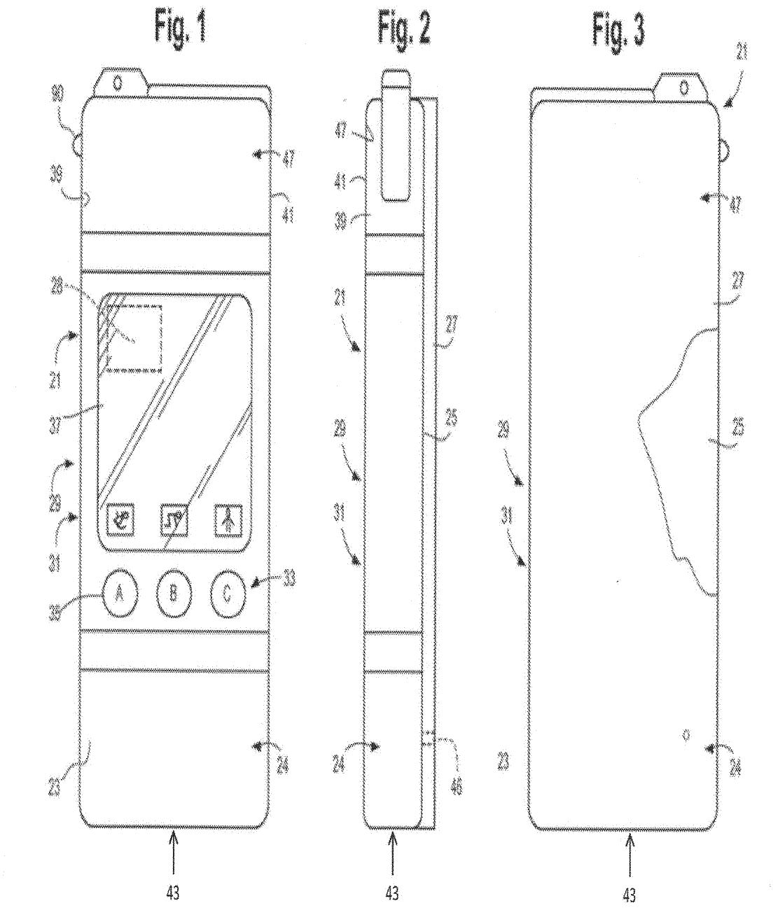

As best seen in FIG. 2, interface module 31 may be configured so as to connect to power module 47 at a first location and sensor module 24 at a second location on module 31. In this embodiment, respective surfaces of modules 24, 31, and 47 form contact surface 25, to which a layer of gel material 27 is applied having a thickness of about ⅛ inch to ¼ inch. The configurations of contact surface 25 and gel material 27 may be varied depending on the application or device form-factors desired. The weight of device 21, as transmitted to contact surface 25, as well as the thickness or tackiness of gel material 27, are “tuned” or otherwise selected so that sensor module 24 is in operative contact with the child being diagnosed, remains stable for a clinically sufficient period of time so that diagnosis is accomplished reasonably accurately, and is isolated so as to reduce the occurrence of spurious or inaccurate readings during such diagnosis. Gel material 27 in this implementation comprises a layer of silicone configured to be applied to contact surface 25 and selectively peeled away therefrom. The silicone layer is chosen so as to remain substantially intact, washable, and replaceable back on contact surface 25 for reuse. Other implementations may include a cap for either covering gel material 27, or an applicator for adhering a layer of gel material 27 to all or the desired portion of contact surface 25.

In some implementations, a settling in period before commencing sensing or other diagnostics may be desirable, such as a period of time from when the device 21 is placed on the child's chest or other body part to commencement of sensor detection. Control system 29 can likewise be programmed to account for such settling time or otherwise adjust sampling periods or filter sensed input to further assure accurate readings.

The operation of portable, pediatric respiratory rate sensing device 21 may be readily appreciated from the foregoing description. A medical practitioner, aid worker, or other Held personnel may use the device in connection with a child to be examined. After turning on the device through power switch or other suitable means (not shown), a suitable respiratory program is selected, in this case infant, toddler, or child, by depressing corresponding user-selectable buttons 35. Contact surface 25, including gel material 27 disposed thereon, is placed on the child's chest at some point before or after the desired program is selected. The tackiness of gel material 27 permits adherence even if the child is not perfectly still, as may sometimes occur when dealing with children. The resiliently compressible characteristics of gel material 27 may likewise serve to insulate or cushion sensor module 24 from outside shocks or other unintended input, which could result in spurious readings.

Readouts from the diagnostic being performed are suitably displayed on screen 37. Depending on the particular application, such readouts can be numeric, graphic, include sounds, lights, or other suitable indicators, in any suitable combination to indicate the respiratory rate.

In this implementation, simultaneous with detection of respiration by means of accelerometer 23, or as an alternative thereto, temperature sensor 45 may detect the child's temperature through suitable operative contact with the child, and a corresponding reading can be displayed in screen 37, whether numeric, color-coded, or audio signal in nature.

If the layer of silicone on device 21 becomes dirty or otherwise loses sufficient tackiness to operatively contact the child being diagnosed, the user may peel the used gel material away from contact surface 25 and cither replace it with another silicone film, or wash the soiled silicone film and return it to the contact surface.

Portable, pediatric respiratory rate sensing device 21 is just one possible implementation of a pediatric medical diagnostic device 121 shown schematically in FIG. 4, in which like reference numerals correspond to like components.

As in the case of device 21, pediatric medical diagnostic device 121 includes a sensor module 124 which may be adapted to detect not only respiratory rate or temperature as discussed with reference to the previous implementation, but may include one or more of the following sensors: a galvanic sensor, a heart rate sensor, an oximeter sensor, a stethoscope, a flow meter, an ECG/EKG sensor, an otoscope, or a photographic camera. The foregoing sensors may be housed separately in respective sensor modules, or may be combined together into one or more sensor modules, as appropriate. It will likewise be appreciated that, depending on the sensor, operative contact with different body locations of the child is contemplated. In this way, a plurality of the sensor modules 124, including those specified above, may be removably connected to a suitably multiplexed version of sensor connector 32, so as to electrically connect to control system 129 of device 121. Sensor connector 32, in this implementation, is thus suitably adapted to removably receive any selected one of sensor modules 124, so that device 121 can be used to diagnose any number of conditions of a child to be examined.

Sensor modules 124 and control system 129 include suitable programming, interfaces, connectors, or drivers to appropriately receive inputs from the child being examined, or from the selected one or more sensor modules 124, and process such inputs. Programming also allows the user to select corresponding programs through user interface module 131, and display suitable readouts through screen 137.

Medical diagnostic device 121, in this implementation, is hand-held, and includes a contact surface coated with gel material, as well as a portable, rechargeable power source electrically connected to user interface module 131, as discussed with reference to device 21. As in device 21, medical diagnostic device 121 is configured so that its area and weight associated with its contact surface, as well as the characteristics of its gel material, lead to substantial stabilization of the sensor module 124 during use.

Devices 21, 121 may include a transmitter, such as that shown at 141 in FIG. 4. Transmitter 141 is operatively connected to control system 29, 129 through a suitable communication interface 143. In cither application, transmitter 141 sends signals corresponding to the condition detected by accelerometer 33 or the corresponding sensor module 24, 124 in a suitable transmission format, using a suitable antenna 148 as needed. Devices 21, 121 may make use of Bluetooth, Wi-Fi, NFC, radio, cellular, AM/FM, 802.5.14, or other suitable protocols for wireless diagnostic devices.

Devices 21, 121 may be equipped with a suitable LED 90, 190 to illuminate surroundings. Devices 21, 121 are suitably equipped not only with processing capabilities, drivers, and other software programming to operate sensor modules as discussed above, but likewise include suitable internal memory, such as flash/EPROM and SDRAM, as well as removable memory in the form of MMC/SD cards. As such, diagnostics procedures performed on one or more children may not only be transmitted by the means discussed previously, but may be stored in suitable memory for uploading or removed for use by other systems.

In a similar vein, devices 21, 121 are equipped with a suitable connection interface, such as a USB port 191, which may be used not only to charge power module 47, 147 therein, but to upload or transfer data to a computer device or computer network.

Although device 21 has been shown to include a substantially rectangular contact surface 25, it will be appreciated that any number of variations to the form of contact surface 25, as well as the overall form of device 21, are contemplated within the scope of the present disclosure. Thus, for example, in one alternative implementation, in portable medical diagnostic device 21, gel material 27 may be suitably applied to lower surfaces of a pair of resiliently flexible straps 245, as shown in FIG. 5. Straps 245 extend from housing 243, which may carry one or more of the sensors discussed previously, a suitable portable power source, and the associated user interface discussed with reference to devices 21, 121. The straps 245 may be extended so as to be in operative contact with the appropriate location on the child's body being diagnosed, such as the chest in the case of an accelerometer. The form factor of device 221 shown in FIG. 5 may be such as to be worn around either the user's wrist or the patient's wrist, with straps 245 being flexible enough wrap around and be retained on such wrist.

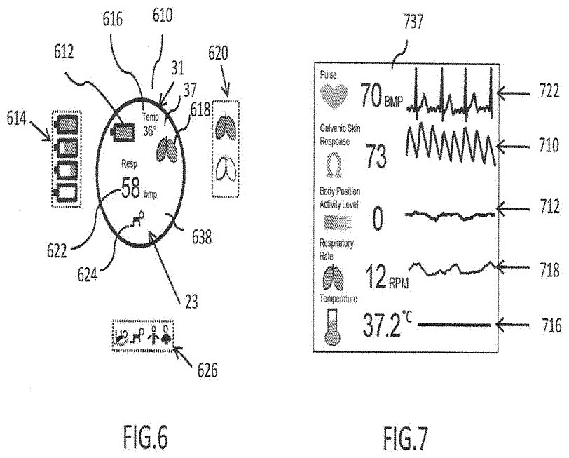

FIG. 6 presents one implementation of the sensor module. To advance the implementation for a settling in period before sensing or other diagnostics the sensing device can employ various sensing conditions to validate an initiation for the engagement of a secondary or tertiary sensing condition. For example, the device can be moved from the wrist of a healthcare worker and placed mid thorax onto a pediatric patient. As shown in FIG. 6, in one embodiment, the sensor module 610 can be initiated to begin a diagnostic protocol by starting with two accelerometers 23 implanted in the device 21: one to measure body position: flat or supine, sitting, standing, or rotated on either side; the other accelerometer to measure body activity; vibration from speaking, convulsing, tremors, jitters, jimmy-leg, or other anomalous movements. Should the body be measured for any of the aforementioned conditions then various other sensors would be employed for any number of protocols that would lead to a proper or validated diagnostic measure and express those measurements in device 21. As shown in FIG. 6, vital sign readouts are displayed in the user interface module 31 further includes a user touchscreen 638. A user can select vital sign program by touching user input area on the touchscreen 638 and the corresponding readouts are displayed on the screen 37. For example, a battery state readout 612 is measured by a battery state sensor in the sensor module 610. Four battery states indicating different battery levels are shown as 614. Wearer's body temperature readout 616 is measure by the temperature sensor 45. Pediatric respiratory rate readout 618 is measured by a respiratory sensor in the sensor module 610. Two respiratory states indicating different respiratory rates are shown as 620. Pulse rate readout 622 is measured by a heart rate sensor in the sensor module 610. Operation mode 624 is selected from four operation modes are shown in 626: infant, toddler, child, and adult modes.

In one embodiment, the sensor module includes a communicating module and a transmitting module. By way of example, the communicating module includes one or more networks such as a data network, a wireless network a telephony network, or any combination thereof. It is contemplated that the data network may be any local area network (LAN), metropolitan area network (MAN), wide area network (WAN), the Internet, or any other suitable packet-switched network, such as a commercially owned, proprietary packet-switched network, e.g., a proprietary cable or fiber-optic network. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wireless fidelity (WiFi), satellite, mobile ad-hoc network (MANET), and the like.

Furthermore, the transmitting module may include, for example, coaxial cables, copper wire, fiber optic cables, and carrier waves that travel through space without wires or cables, such as acoustic waves and electromagnetic waves, including radio, optical and infrared waves.

The sensor array measurements can be transmitted remotely to a more advanced receiver to analyze, compare and compartmentalize the results from the device 21. The sensor data comprises the plurality of sensor input under a format that represents each sensor's measurements correlated to the other sensor's measurements across time. The result when viewed or transmitted will be the condition of the body (supine, erect, sitting, etc.), the activity of the body (convulsing, girding, coughing, or other activities other than body position), location (geospatially) of the body, environmental measurements external to the body, air quality external to the body, and all other external, internal measurements of the body and the environment the body presents. The data format provides the viewer or receiver the ability to review the time the measurements were taken in a sparkline or other graphical format to assess each measurement in comparison, coordination, compilation, protocol, state of presentation to assist the appropriate receiver to make a clear diagnosis or prognosis based on the matrix of information presented. The sensor array measurements can be held for transmission to a remote device when the sensor array has been removed from the body. For example, as shown in FIG. 7, vital sign readouts are displayed as a sensor array in a display screen 737. For example, pulse rate 722, Galvanic skin response 710, body position activity level 712, pediatric respiratory rate 713, and body temperature 712 are displayed, according to one example embodiment.

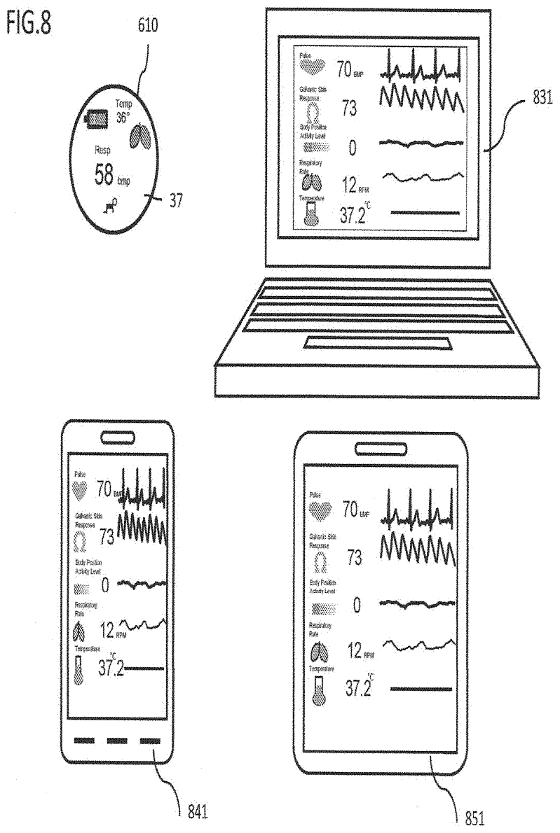

FIG. 8 shows some more vital sign readouts display examples. The vital sign readouts displays on screen 37 of the sensor module 610 can be transmitted to and analyzed at a computer 831, a mobile phone 841, and/or a tablet 851. The vital sign results can be transmitted from one device to another through Bluetooth, Wi-Fi, NFC, radio, cellular, AM/FM, 802.5.14, or other suitable protocols for wireless diagnostic devices.

Another example relates to diabetes where a pediatric patient may place the device onto a body part, e.g. upper arm, mid-thorax, upper thigh, around the neck or across the back of the body. The pediatric patient would then go about their daily routine; as they walk the sensing device would use various sensor measured conditions to initiate other sensor's initiation, within this example, sensing conditions for walking would initiate heart rate and respiratory rate sensing with galvanic skin response, whereas, while the body is at rest the heart rate and respiratory rate would but the accelerometers into a sleep condition. From a day, week or month of monitoring, the results from each sensor would be viewed as an array to make a protocol for various health related conditions.

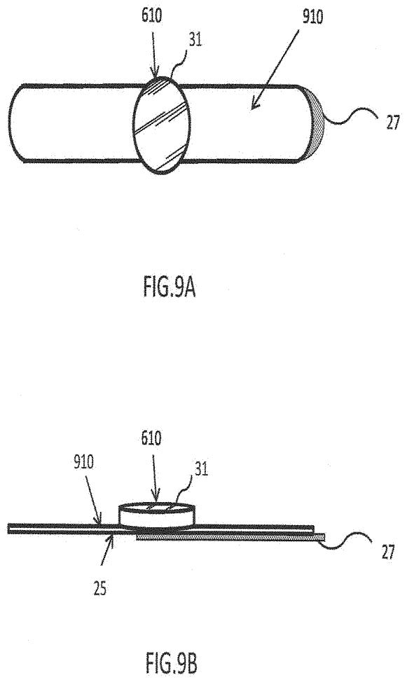

As shown in FIG. 9A-B, the provision of the current invention provides types of ends to the banding system where various types of sensors, devices, functions are provisioned. FIG. 9A is the front view of the sensor module 610 on a banding system 910. In one example, the banding system 910 includes a contact surface 25 to make contact at with a wearer's body to relay bio-electrical signals to the sensor module. The user interface module 31 having a lower surface at least partially comprising the contact surface 25. In another example, gel material 27 is at least partially coating the contact surface 25. The gel material 27 is made of silicone and rubberized carbon electrodes. The silicone provides flexibility of use and carbon electrodes adhere to the silicone banding system and collect bio-electrical impulses from the body. The rubberized carbon electrodes are intermittently placed across the banding system 910 to make contact at various places on the body and to relay the body electrical impulses to various sensors. FIG. 9B is the side view of the sensor module 610 on the banding system 910.

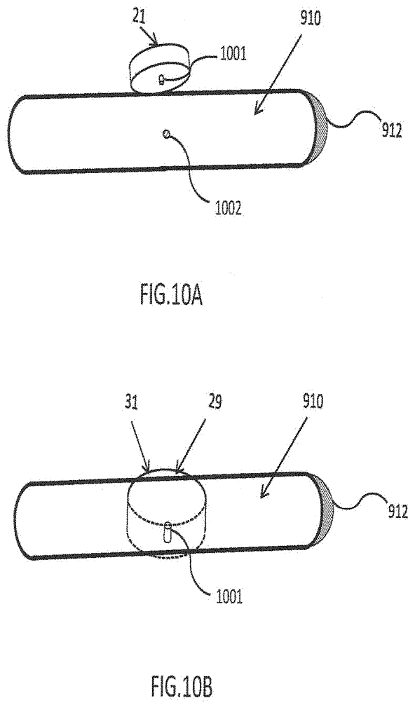

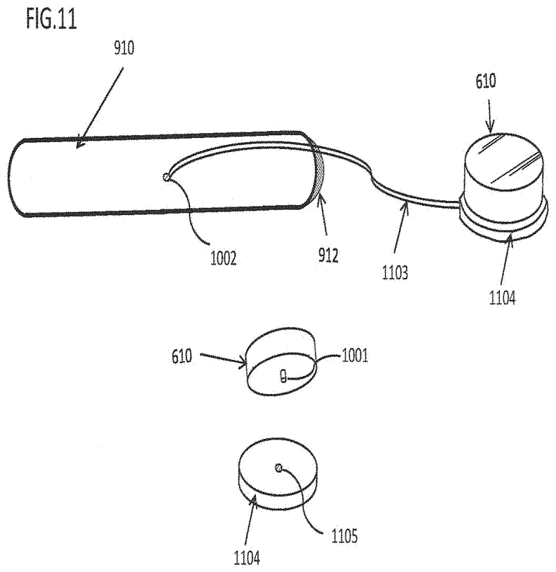

FIGS. 10A-B display steps for attaching the sensor module 610 to the banding system 910. Sensor module 610 has male/female connecting mechanism. As a way of example, FIG. 10A shows the sensor module 610 has a male connecting mechanism 1001. Banding system 910 has a receiving male/female connecting mechanism. As a way of example, FIG. 10A shows that there is a female connecting mechanism 1002 on the banding system 910. The sensor module 610 can be attached to and removed from the banding system 910 by connecting and de-connecting the male/female connecting mechanism (as shown in FIG. 10B). The banding system 910 can sense bio-electrical impulses from the wearer's body through rubberized carbon electrodes on the banding system 910 and relay the bio-electrical impulses to the sensor module 610 through the connection between the sensor module 610 and the banding system 910.

As shown in FIG. 11, in another embodiment, the sensor module 610 is connected to the banding system 910 through an extension cable 1103. By way of example, as shown in FIG. 11, the sensor module 610 has a male connecting mechanism 1001. A base 1104 has a receiving female connecting mechanism 1105. The sensor module 610 and the base 1104 can be connected through the male/female connecting mechanisms 1001 and 1105 and then the base can be connected to the banding system 910 through the extension cable 1103. The bio-electrical impulses sensed by the banding system 910 can be transmitted to the sensor module through the extension cable 1103.

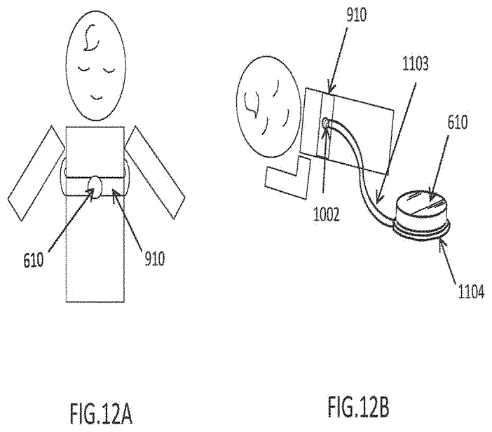

FIG. 12A-B present two wearing modes of the device 21. FIG. 12A represents a direct wear mode. The wearer can wrap the banding system around a body part. The banding system 910 comprises layered, flexible bistable spring material, which are sealed within a silicone and rubberized carbon electrode material or other material used for the sensing of bio-electrical impulse signals from wearer's body. The banding system 910 can be straightened out (as shown in FIG. 5), making tension within springy band of various materials, commonly steel or plastic. Then the straightened banding system can slap against the wearer's body part, causing the band to spring back in to curve that wraps around the body part, securing the device to wear. FIG. 12B represents an indirect wearing mode wherein an extension cable 1103 separates the sensor module 610 and the banding system 910 while maintaining operational system status.

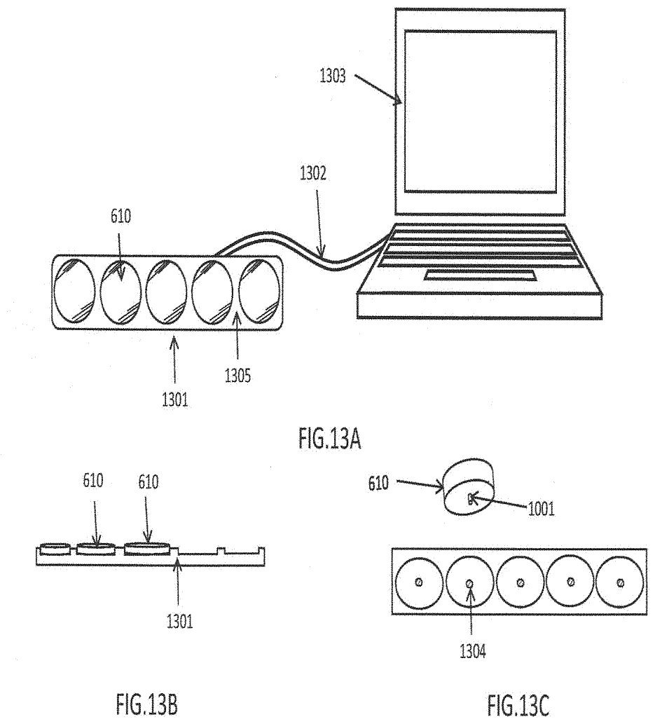

As shown in FIG. 13A-C, in one embodiment, the sensor modules 610 are charged in a charging system 1301. In one example, the charger is a crank charger. The charging system has a charging strip 1305. The sensor module 610 has male/female connecting mechanism. As a way of example, FIG. 13C shows the sensor module 610 has a male connecting mechanism 1001. The charging system 1301 has a receiving male/female connecting mechanism. As a way of example, FIG. 13C shows that there is a female connecting mechanism 1304 on the charging system 1301. The sensor module 610 can be attached to and removed from the charging system 1301 by connecting and de-connecting the male/female connecting mechanism. The charging system 1301 can charge multiple sensor modules at a single time, as shown in FIG. 13B. The charging system 1301 is connected to a computer 1303 through an extension cable 1302. The computer 1303 can provide power source to the charging system. Vital signs sensed by various sensors in the sensor module 610 and by rubberized carbon electrodes intermittently placed across the banding system 910 can also be transmitted to the computer 1303 through the extension cable 1302.

The present invention has the following capabilities; where contact leads extend from the center of the device to each end where the following devices, sensors or functionality are engaged:

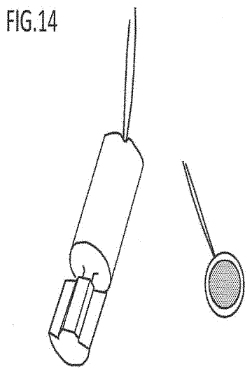

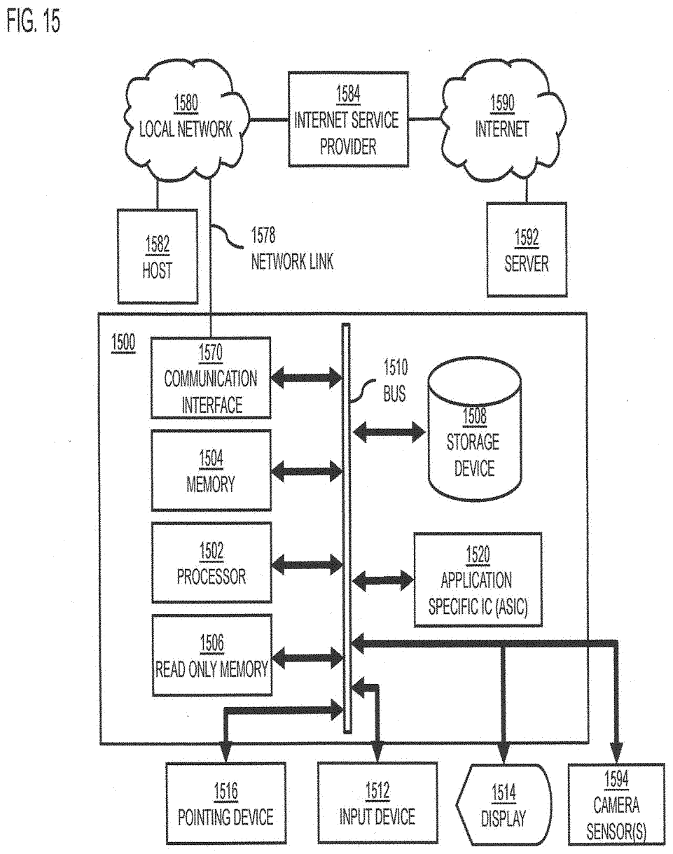

Vibration: Engaged from rubberized carbon electrodes measuring vital signs to the sensor module 610 engage the vibration device(s) (as shown in FIG. 14) at each end in a system to tickle, nudge, startle the patient to breath or come to awareness of the vibration; and

Defibrillator: Engaged from the rubberized carbon electrodes measuring vital signs to the device 21 engage the defibrillator; and

Microphone/Speaker: Engaged from the rubberized carbon electrodes measuring vital signs, sound amplified from external to the wearer's body, sound amplified internal to the body, and sound from speaker to the body and sound from speaker to the environment; and

Reactive Memory Banding Material: Engaged from the rubberized carbon electrodes measuring vital signs to the device 21 engage reactive memory banding material to provide various levels of extension and contraction moving the banding material alternately between the sides of the band or in unison; and

Medication dispensing: the device 21 of the present invention is a fabrication of high-performance, energy-efficient sensors and memory modules that are in intimate mechanical contact with soft tissues of the body where successfully “herding” groups of cells using electrical currents, heat, vibration or a combination in conjunction with the controlled delivery of therapeutic agents, provides for the delivery of various medications to the body for pneumonia, topical conditions and many other conditions requiring the dispensing of medication from the points of contact where the banding system 910 is in contact to the body. The banding system 910 through the rubberized carbon electrodes and force of the tension within the springy bands of various materials, commonly steel or plastic, adhere to the body as tacky material but not adhered to the body.

In another embodiment, the present invention can provide private medical information content to a voice telephone call parties prior to, during or following a call. In one aspect, a system and method may be implemented in a voice telephone communication apparatus that is adapted to receive and hold information content from various sensor or transmitted sources on behalf of the call party, gather information that is private to the call party and add it to the information store as part of the information content, and retrieve the information content from the information store and present it to the call party in response to an information presentation initiation action. In another aspect, the system cooperates with health related equipment or person on premises or medical equipment monitoring system, such as equipment whose purpose is to measure the state of being of a body for medical and health purposes in a system, to provide health related monitoring information to a telephone call party. For example, as shown in FIG. 4, devices 21, 121 include a transmitter, which is operatively connected to control systems 29, 129 through a suitable communication interface 143. The control system 29, 129 are programmed to process input from the user interface 143 on device 21, 121 and remotely to device 21, 121 from a remote voice telephone for all sensing sensors in the sensor module 610 and the banding system 910. The transmitter sends signals corresponding to vital signs in a format selected from a group consisting of Bluetooth, Wi-Fi, NFC, radio, cellular, AM/FM, 802.5.14, and protocols of wireless diagnostic devices

The provision of private information to voice telephone parties may be provided via a set of communications standards for simultaneous digital transmission of voice, video, data, and other network services over the traditional circuits of the public switched telephone network. The information may be provisioned prior to, during or following a call.

Embodiments can collect vital sign information from a patient/wearer's body and transit the data to a user's cellphone or a portable pediatric medical diagnostic device.

On a portable pediatric medical diagnostic device, a software application can stage the information for transmission, i.e., recorded data may wait for a unique telephone number to be prepended or appended to the data before it is transmitted.

There are four parties in this communication system: (1) the portable pediatric medical diagnostic device; (2) a patient's cellphone, embodiment, or other communications device; (3) a doctor's cellphone, embodiment, or other communications device; and (4) a hospital's cellphone, embodiment, or other communications device.

Embodiments can transmit information under three formats: (1) prior to call connection or during the ringing; (2) during the call connection; and (3) after both parties disconnect the voice call.

During each of these transmissions, embodiments of the present invention can provide the phone number “from” and “to” a desired party. Therefore, the patient's number is known and identifiable to the doctor and the doctor's number is identifiable to the Patient.

The HIPPA Privacy Rule requires covered healthcare providers to apply reasonable safeguards when making these communications to protect the information from inappropriate use or disclosure. These safeguards may vary depending on the mode of communication used. For example, when faxing protected health information to a telephone number that is not regularly used, a reasonable safeguard may involve a provider first confirming the fax number with the intended recipient.

Similarly, a covered entity may pre-program frequently used numbers directly into the fax machine to avoid misdirecting the information. When discussing patient health information orally with another provider in proximity of others, a doctor may be able to reasonably safeguard the information by lowering his or her voice.

Since fax machines are both analog and digital, point-to-point and use a database of “known” telephone numbers they are allowed from the HIPPA rule of privacy.

Embodiments of the present invention may use the same typology except that sensor information has been assembled and transmitted point-to-point, prior to, during, or after an initiated call.

To increase privacy, the doctor's office may or may not need all embodiments of the invention. Small doctor offices could utilize their current communications system; whereas, larger offices could benefit from having an embodiment of the invention at the doctor's office.

Data flow between patient, doctor and dlinic̨ or hospital: Embodiments of the invention may be placed on a patient/child/wearer, and vital measurements may be taken and stored in the device until polled through Bluetooth into the transmitting device (Cellphone, embodiment of the invention).

The Vital Measurements (VM) may be stored in a software application on an embodiment until the application is engaged to transmit the VM to a doctor or hospital that participates in diagnostic services using the embodiments.

Security: Embodiments of the invention can apply three levels of validation and security to the Vital Measurements data:

On the portable pediatric medical diagnostic device, embodiments can create a hash (a number sequence) that is dynamically changing until a VM is taken. The hash is then passed to an application in a cellphone or embodiment that compares the hash against three validation schemes in the device that receives VM from a registered embodiment. There are only two devices in this example: the portable pediatric medical diagnostic device and the cellphone application that receives the VM local to the patient's body.

The Patient now has the VM in a cellphone application and initiates a transfer to a Doctor or Hospital. The Patient identifies through the application, the person from which the VM came. A doctor's icon that represents a doctor's cellphone is selected and a call is placed along with the hash.

The doctor or hospital can have an embodiment of the present invention installed at the doctor's office or hospital. This embodiment can be either Software or Hardware based, but it must be connected to the doctor's or hospital's telephone system or cellular phone. A doctor's system can receive a call along with the hash that is registered between the Sender (patient) and Receiver (doctor). Software on the calling device can append the hash and place a copy of the VM into the Sender's device. The VM data can then be displayed on the Doctor's device. The period of VM can be played forward or backwards while displaying the relative information for heart rate, respiratory rate, temperature, body position, body activity, anxiety level and device health. The doctor can view the patient's name but the VM data is not labeled in any way that can be identified by human inspection. However, embodiments can validate the name of the patient, the device it was transmitted from and to through the hash within each of the devices.

Relatively similar transmission takes place between the doctor and the hospital with the inclusion of the doctor's hash recognized by embodiments residing at a hospital.

With full security and point-to-point transmission, the entire communications process falls within the specifications of HIPPA.

Liability: The level of liability is directly proportional to the strength of adoption and the validation of the VM transmission protocol through the publication of clinical data from NGO/Commercialized market sources.

The processes described herein for providing detect of one or more corresponding medical conditions of a wearer may be advantageously implemented via software, hardware (e.g., general processor, Digital Signal Processing (DSP) chip, an Application Specific Integrated Circuit (ASIC), Field Programmable Gate Arrays (FPGAs), etc.), firmware or a combination thereof. Such exemplary hardware for performing the described functions is detailed below.

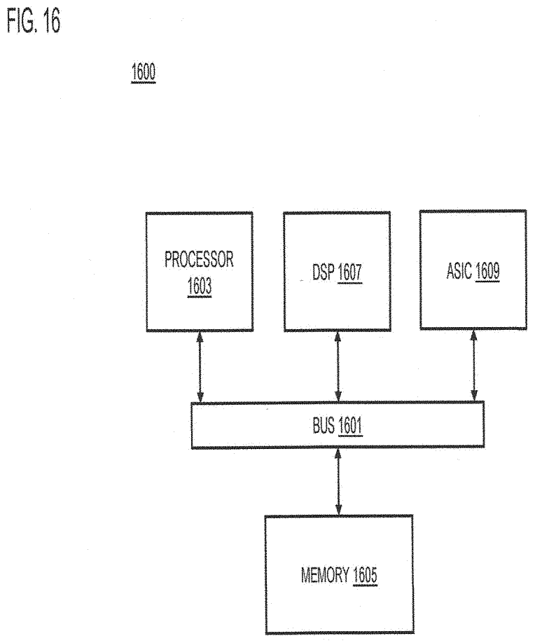

FIG. 16 illustrates a computer system 1600 upon which an embodiment of the invention may be implemented. Computer system 1600 is programmed (e.g., via computer program code or instructions) to detect one or more corresponding medical conditions of a wearer as described herein and includes a communication mechanism such as a bus 1610 for passing information between other internal and external components of the computer system 1600. Information (also called data) is represented as a physical expression of a measurable phenomenon, typically electric voltages, but including, in other embodiments, such phenomena as magnetic, electromagnetic, pressure, chemical, biological, molecular, atomic, sub-atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range.

A bus 1610 includes one or more parallel conductors of information so that information is transferred quickly among devices coupled to the bus 1610. One or more processors 1602 for processing information are coupled with the bus 1610.

A processor 1602 performs a set of operations on information as specified by computer program code related to detect one or more corresponding medical conditions of a wearer. The computer program code is a set of instructions or statements providing instructions for the operation of the processor and/or the computer system to perform specified functions. The code, for example, may be written in a computer programming language that is compiled into a native instruction set of the processor. The code may also be written directly using the native instruction set (e.g., machine language). The set of operations include bringing information in from the bus 1610 and placing information on the bus 1610. The set of operations also typically include comparing two or more units of information, shifting positions of units of information, and combining two or more units of information, such as by addition or multiplication or logical operations like OR, exclusive OR (XOR), and AND. Each operation of the set of operations that can be performed by the processor is represented to the processor by information called instructions, such as an operation code of one or more digits. A sequence of operations to be executed by the processor 1602, such as a sequence of operation codes, constitute processor instructions, also called computer system instructions or, simply, computer instructions. Processors may be implemented as mechanical, electrical, magnetic, optical, chemical or quantum components, among others, alone or in combination.

Computer system 1600 also includes a memory 1604 coupled to bus 1610. The memory 15604, such as a random access memory (RAM) or other dynamic storage device, stores information including processor instructions for detecting one or more corresponding medical conditions of a wearer. Dynamic memory allows information stored therein to be changed by the computer system 1600. RAM allows a unit of information stored at a location called a memory address to be stored and retrieved independently of information at neighboring addresses. The memory 1604 is also used by the processor 1602 to store temporary values during execution of processor instructions. The computer system 1600 also includes a read only memory (ROM) 1606 or other static storage device coupled to the bus 1610 for storing static information, including instructions, that is not changed by the computer system 1600. Some memory is composed of volatile storage that loses the information stored thereon when power is lost. Also coupled to bus 1610 is a non-volatile (persistent) storage device 1608, such as a magnetic disk, optical disk or flash card, for storing information, including instructions, that persists even when the computer system 1600 is turned off or otherwise loses power.

Information, including instructions for detecting one or more corresponding medical conditions of a wearer, is provided to the bus 1610 for use by the processor from an external input device 1612, such as a keyboard containing alphanumeric keys operated by a human user, a sensor, a microphone, an Infrared (IR) remote control, a joystick, a game pad, a stylus pen, or a touch screen. A sensor detects conditions in its vicinity and transforms those detections into physical expression compatible with the measurable phenomenon used to represent information in computer system 1600. Other external devices coupled to bus 1610, used primarily for interacting with humans, include a display device 1614, such as a cathode ray tube (CRT), a vacuum fluorescent display (VFD), a liquid crystal display (LCD), a light-emitting diode (LED), an organic light-emitting diode (OLED), a quantum dot display, a virtual reality (VR) headset, or plasma screen or printer for presenting text or images, and a pointing device 1616, such as a mouse, a trackball, cursor direction keys, or motion sensor, for controlling a position of a small cursor image presented on the display 1614 and issuing commands associated with graphical elements presented on the display 1614. In some embodiments, for example, in embodiments in which the computer system 1600 performs all functions automatically without human input, one or more of external input device 1612, display device 1614 and pointing device 1616 is omitted.

In the illustrated embodiment, special purpose hardware, such as an application specific integrated circuit (ASIC) 1620, is coupled to bus 1610. The special purpose hardware is configured to perform operations not performed by processor 1602 quickly enough for special purposes. Examples of ASICs include graphics accelerator cards for generating images for display 1614, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 1600 also includes one or more instances of a communications interface 1670 coupled to bus 1610. Communication interface 1670 provides a one-way or two-way communication coupling to a variety of external devices that operate with their own processors, such as printers, scanners and external disks. In general the coupling is with a network link 1678 that is connected to a local network 1680 to which a variety of external devices with their own processors are connected. For example, communication interface 1670 may be a parallel port or a serial port or a universal serial bus (USB) port on a personal computer. In some embodiments, communications interface 1670 is an integrated services digital network (ISDN) card or a digital subscriber line (DSL) card or a telephone modem that provides an information communication connection to a corresponding type of telephone line. In some embodiments, a communication interface 1670 is a cable modem that converts signals on bus 1610 into signals for a communication connection over a coaxial cable or into optical signals for a communication connection over a fiber optic cable. As another example, communications interface 1670 may be a local area network (LAN) card to provide a data communication connection to a compatible LAN, such as Ethernet. Wireless links may also be implemented. For wireless links, the communications interface 1670 sends or receives or both sends and receives electrical, acoustic or electromagnetic signals, including infrared and optical signals, that carry information streams, such as digital data. For example, in wireless handheld devices, such as mobile telephones like cell phones, the communications interface 1670 includes a radio band electromagnetic transmitter and receiver called a radio transceiver. In certain embodiments, the communications interface 1670 enables connection for detecting one or more corresponding medical conditions of a wearer by the device 21.

The term computer-readable medium is used herein to refer to any medium that participates in providing information to processor 1602, including instructions for execution. Such a medium may take many forms, including, but not limited to, non-volatile media, volatile media and transmission media. Non-volatile media include, for example, optical or magnetic disks, such as storage device 1608. Volatile media include, for example, dynamic memory 1604. Transmission media include, for example, coaxial cables, copper wire, fiber optic cables, and carrier waves that travel through space without wires or cables, such as acoustic waves and electromagnetic waves, including radio, optical and infrared waves. Signals include man-made transient variations in amplitude, frequency, phase, polarization or other physical properties transmitted through the transmission media. Common forms of computer-readable media include, for example, a floppy disk, a flexible disk, hard disk, magnetic tape, any other magnetic medium, a CD-ROM, CDRW, DVD, any other optical medium, punch cards, paper tape, optical mark sheets, any other physical medium with patterns of holes or other optically recognizable indicia, a RAM, a PROM, an EPROM, a FLASH-EPROM, EEPROM, a flash memory, any other memory chip or cartridge, a carrier wave, or any other medium from which a computer can read.

FIG. 17 illustrates a chip set 1700 upon which an embodiment of the invention may be implemented. Chip set 1700 is programmed to detect one or more corresponding medical conditions of a wearer as described herein and includes, for instance, the processor and memory components described with respect to FIG. 16 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip.

In one embodiment the chip set 1700 includes a communication mechanism such as a bus 1701 for passing information among the components of the chip set 1700. A processor 1703 has connectivity to the bus 1701 to execute instructions and process information stored in, for example, a memory 1705. The processor 1703 may include one or more processing cores with each core configured to perform independently. A multi-core processor enables multiprocessing within a single physical package. Examples of a multi-core processor include two, four, eight, or greater numbers of processing cores. Alternatively or in addition, the processor 1703 may include one or more microprocessors configured in tandem via the bus 1701 to enable independent execution of instructions, pipelining, and multithreading. The processor 1703 may also be accompanied with one or more specialized components to perform certain processing functions and tasks such as one or more digital signal processors (DSP) 1707, or one or more application-specific integrated circuits (ASIC) 1709. A DSP 1707 typically is configured to process real-world signals (e.g., sound) in real time independently of the processor 1703. Similarly, an ASIC 1709 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1703 and accompanying components have connectivity to the memory 1605 via the bus 1701. The memory 1705 includes both dynamic memory (e.g., RAM, magnetic disk, writable optical disk, etc.) and static memory (e.g., ROM, CD-ROM, etc.) for storing executable instructions that when executed perform the inventive steps described herein to detect one or more corresponding medical conditions of a wearer. The memory 1705 also stores the data associated with or generated by the execution of the inventive steps.

The described and illustrated arrangements are intended to provide a general understanding of the structure of various embodiments, and they are not intended to serve as a complete description of all the elements and features of the devices and related methods herein. Many other arrangements will be apparent to those of skill in the art upon reviewing the above description. Other arrangements may be utilized and derived therefrom, such that structural and logical substitutions and changes may be made without departing from the spirit and scope of this disclosure. Figures are also merely representational or, as indicated, schematic, and thus may not be drawn to scale. Certain proportions thereof may be exaggerated, while others may be minimized. Accordingly, the specification and drawings are to be regarded in illustrative rather than a restrictive sense.

1. A portable, pediatric vital signs sensing device comprising: a sensor module that connects to a banding system and to an communication system to support an operation of the pediatric vital signs sensing device, wherein the banding system includes a contact surface to make contact at with a wearer's body to relay bio-electrical signals to the sensor module; and wherein the sensor module comprises at least one of an accelerometer, a temperature sensor, a respiratory rate sensor, a galvanic skin sensor, a heart rate sensor, a pulse oximeter sensor, a battery state sensor, a communicating module, and a transmitting module.

2. The device of claim 1, wherein the sensor module further comprises a male/female connecting mechanism to connect the sensor module to a receiving male/female location placed on the banding system; wherein the sensor module is removable from the banding system; and wherein one or more rubberized carbon electrodes are intermittently placed across the banding system to make contact with the wearer's body.

3. The device of claim 2, wherein the one or more rubberized carbon electrodes intermittently placed across the banding system relay electrical impulses from the wearer's body to the sensor module.

4. The device of claim 1, further comprising: an extension cable to separate the sensor module from the banding system while maintaining an operational system status.

5. The device of claim 1, further comprising a charging system for charging the sensor module, wherein the sensor module further comprises a male/female connecting mechanism to connect the sensor module to a receiving male/female location placed on the charging system; wherein the sensor module is removable from the charging system; and wherein the charging system can charge multiple sensor modules at a single time.

6. The device of claim 1, wherein the device is configured to sense vital signs in all age groups.

7. The device of claim 1, further comprising a control system for the device, wherein the control system comprises: a user interface and a processor that process a local input from the user interface on the device, a remote input sent to the device from a remote calling unit, or a combination thereof for all sensors in the device.

8. The device of claim 7, wherein the control system operates the device in multiple modes for all age groups.

9. The device of claim 1, further comprising: a transmitter operatively connected to the sensor module to send signals corresponding to vital signs detected by the sensor module in a format selected from a group consisting of Bluetooth, Wi-Fi, NFC, radio, cellular, AM/FM, 802.5.14, and protocols of wireless diagnostic devices.

10. The device of claim 1, further comprising: a user interface in the sensor module having a user touchscreen input area to select a vital sign program and a screen for displaying a corresponding readout; and a first sensor module including the sensor module, and a first power module including a rechargeable power source, wherein the first power module and the first sensor module are user-detachable to enable attachment of a second power module and a second sensor module; wherein the second power module and the second sensor module are different from the first power module and the first sensor module; and wherein the first sensor module and the second sensor module can be charged together on a charging strip in a charging system where the first sensor module and the second module can be connected for charging.

11. The device of claim 10, wherein the screen comprises a touchscreen and at least some of the user input areas are available via the touch screen, a user interface module having a lower surface at least partially comprising the contact surface.

12. The device of claim 10, wherein the user touchscreen input area comprises one or more selection buttons corresponding to respective modes of operation selected from all age groups.

13. The device of claim 1, wherein the temperature sensor operatively connected to the contact surface.

14. The device of claim 1, wherein the banding system comprises: a layered, flexible spring material, sealed within a silicone and rubberized carbon electrode material or other material used for sensing of the bio-electrical signals from the wearer's body, wherein the banding system can be straightened out under tension and wrapped around the wearer's body when not under tension to secure the device to the wearer's body.

15. A hand-held pediatric medical diagnostic device, comprising: a sensor module adapted to detect one or more corresponding medical conditions of a wearer, wherein the sensor module includes at least one sensor selected from the group consisting of an accelerometer, a thermometer, a galvanic sensor, a heart rate sensor, an oximeter, a stethoscope, a flow meter, an ECG/EKG sensor, an otoscope, and a photographic camera; a contact surface of a banding system operatively connected to the sensor module, the contact surface adapted to be placed in operative contact with the wearer; a control module including a user interface having user input areas to initiate at least one diagnostic program associated with the sensor module, and a processor suitably programmed to process input from the user interface and from the sensor module; a portable, rechargeable power source electrically connected to the control module.

16. The device of claim 15, further comprising: a gel material at least partially coating the contact surface of the banding system, wherein the gel material is made of silicone and a rubberized carbon material; and wherein the rubberized carbon electrodes adhere to the silicone and collect bio-electrical impulse signals from the wearer's body.

17. The device of claim 16, wherein the contact surface has an area about 10 square inches to about 14 square inches, a weight of about 130 grams to about 200 grams, and the gel material has an area substantially corresponding to the contact surface, a thickness of about ⅛ inch, and a tackiness sufficient, under standard atmospheric conditions, to adhere to the skin of the wearer being examined and to be removable therefrom.

18. The device of claim 15, further comprising: a power module to provide power for the device, wherein the power module can be recharged, disconnected from the charger, and put into use.

19. The device of claim 15, further comprising a transmitter operatively connected to a control system to send signals corresponding to a medical condition detected by the sensor module in a format selected from the group consisting of Bluetooth, Wi-Fi, NFC, radio, cellular, AM/FM, 802.5.14, and protocols of wireless diagnostic devices.

20. The device of claim 15, further comprising a plurality of the sensor modules and a multiplexed sensor connector electrically connected to a control system, wherein the connector is adapted to removably receive any selected one of the plurality of the sensor modules.
